(19) United States
(12) Patent Application Publication
(10) Pub. No.: US 2019/0254566 A1
(43) Pub. Date: Aug. 22, 2019

(54) SYSTEMS AND METHODS FOR ESTIMATION OF RESPIRATORY MUSCLE PRESSURE AND RESPIRATORY MECHANICS USING P0.1 MANEUVER

(71) Applicant: KONINKLIJKE PHILIPS N.V., EINDHOVEN (NL)

(72) Inventors: ANTONIO ALBANESE, MISSION VIEJO, CA (US); ROBERTO BUIZZA, MALDEN, MA (US); FRANCESCO VICARIO, BOSTON, MA (US)

(21) Appl. No.: 16/345,398

(22) PCT Filed: Oct. 23, 2017
(86) PCT No.: PCT/IB2017/056562

§ 371 Date: Apr. 26, 2019

Related U.S. Application Data
(60) Provisional application No. 62/412,927, filed on Oct. 26, 2016.

(51) Int. Cl. A61B 5/085; A61B 5/03; A61M 16/00; A61B 5/087

(52) U.S. Cl. CPC A61B 5/085; A61B 5/03; A61M 16/022; A61B 5/4836; A61M 2016/0015; A61M 2016/0039; A61B 5/087

(57) ABSTRACT

When estimating respiratory muscle pressure and respiratory mechanics using a P0_{.1} maneuver patient inspiration onset for a patient connected to a ventilator (64) is detected, and the airway of the patient is occluded for a first predetermined time period. A first respiratory muscle pressure (Pₘᵤₛ) profile is estimated during the airway occlusion. Resistance (R) and compliance (C) values and a second Pₘᵤₛ profile generated during a second predetermined time period are then estimated. A third Pₘᵤₛ profile is estimated during a third predetermined time period that extends from the end of the second predetermined time period until the end of inspiration. Pₘᵤₛ(t) over an entire breath is estimated by concatenating the first, second and third Pₘᵤₛ profiles, and the estimated R and C values and the estimated Pₘᵤₛ profiles are output on a display.

### FIELD

The present invention finds application in patient ventilation systems and methods. However, it will be appreciated that the described techniques may also find application in other patient care systems, other patient parameter estimation techniques, and the like.

### BACKGROUND

Estimating respiratory muscle pressure (Pₘᵤₛ(t)) is of paramount importance in support modalities of mechanical ventilation, such as Pressure Support Ventilation (PSV), where patient and ventilator share the mechanical work performed on the respiratory system. Quantitative assessment of Pₘᵤₛ(t) can be used to select the appropriate level of ventilation support in order to prevent both atrophy and fatigue of the respiratory muscles. One clinical parameter commonly used to assess the effort made by the patient per breath is known as Work of Breathing (WOB) and can be computed once the estimate of Pₘᵤₛ(t) is available for the breath (e.g., WOB can be obtained from Pₘᵤₛ(t) by integration of the latter over the inhaled volume). One conventional approach for Pₘᵤₛ(t) and WOB estimation relates to measuring the esophageal pressure (Pₑₛ) via insertion of a balloon-tipped catheter in the patient's esophagus. The measured Pₑₛ(t) is assumed to be a good proxy for the pleural pressure (Pₚₗ) and can be used, in conjunction with an estimate of chest wall compliance, to compute the WoB via the so-called Campbell diagram or, equivalently, via explicit computation of Pₘᵤₛ(t) and then of WOB.

Estimates of R and C are important per se, as they provide quantitative information to the physician about the mechanical properties of the patient's respiratory system and they can be used to diagnose respiratory diseases and better select the appropriate ventilation modalities and therapeutic paths. Moreover, R and C can also be used to estimate Pₘᵤₛ(t) as a non-invasive alternative to the use of the esophageal catheter. Assuming R and C are known, it is indeed possible to estimate Pₘᵤₛ(t) via the following equation (known as the Equation of Motion of the lungs):

P_{aw}(t)=R·\dot{V}(t)+E·V(t)+Pₘᵤₛ(t)+P₀ (1)

where P_{aw}(t) is the pressure measured at the airway opening, \dot{V}(t) is the flow of air into and out of the patient's respiratory system (measured again at the airway opening), V(t) is the net volume of air delivered to the patient (measured by integrating the flow signal over time), E is the elastance (inverse of the compliance C) and P₀ is a constant term to account for the pressure at the end of expiration (needed to balance the equation but not interesting per se).

Previous attempts to use equation (1) for a non-invasive estimation of Pₘᵤₛ(t) relied on a two-step approach, where R and C are estimated first and then equation (1) is applied to compute Pₘᵤₛ(t) using the estimated values of R and C. Estimation of R and C was performed either by applying the End-Inspiratory Occlusion (EIP) maneuver or via Least-Squares (LS) fitting of equation (1) to flow and pressure measurements under specific conditions, where the term Pₘᵤₛ(t) was assumed to be zero. These conditions included:
1. periods of patient paralysis and Continuous Mandatory Ventilation (CMV);
2. periods of high Pressure Support Ventilation (PSV) level;
3. specific portions of every pressure-supported breath that extend both during the inhalation and the exhalation phases;
4. exhalation portions of pressure-supported breaths, where the flow signal satisfies specific conditions that are indicative of the absence of patient inspiratory efforts.

The quantitative assessment of the mechanical properties of the respiratory system and of the inspiratory efforts in patients under mechanical ventilation offers invaluable information for the clinician to tailor ventilation strategy and settings. The current state of the art for the assessment of respiratory mechanics consists of computing two parameters, namely resistance (R) and compliance (C), via the EIP technique. This technique, however, not only interferes with the normal operation of the ventilator, but it requires the respiratory muscles to be fully relaxed in order to provide accurate R and C estimates. Hence, because of the presence of respiratory activity from the patient, the EIP often leads to biased results. Assessment of inspiratory patient efforts, on the other hand, is traditionally obtained by inferring the pressure generated by the respiratory muscles (Pₘᵤₛ) from the pressure measured in the esophagus (Pₑₛ). Quantitative assessment of patient effort is then obtained on a breath-by-breath basis by computing Work of Breathing (WOB) from the Pₘᵤₛ waveform. The main limitation of such approach is that measurement of Pₑₛ requires the insertion of an esophageal catheter, with consequent discomfort for the patient in addition to the need for special instrumentation and skilled personnel.

Other methods have been developed to allow simultaneous estimation of R, C and Pₘᵤₛ(t) from measured airway pressure and flow waveforms during regular ventilation without requiring esophageal pressure measurements. These methods are based on the use of the traditional first-order single-compartment model of respiratory mechanics and its associated Equation of Motion in (1). They all face the fundamental difficulty of the simultaneous estimation approach related to the underdetermined nature of the mathematical problem (more unknowns than available equations). In these methods, the use of constrains based on physiological assumptions have been advocated to render the mathematical problem solvable. However, these methods have been shown to work only under specific conditions. Particularly, when the ventilator cycles off before the patient has completely released his respiratory muscles (i.e., Pₘᵤₛ has returned to zero baseline value), these conventional methods are not reliable. This may limit their applicability to all clinical scenarios.

The disadvantage of the conventional invasive procedure of esophageal pressure measurement is apparent, since the insertion of an esophageal balloon requires experienced personnel and implies discomfort and risk for the patient.

The two-step estimation technique, where the EIP maneuver is first performed to get R and C and equation (1) is then used to compute Pₘᵤₛ(t), has the following main drawbacks:
1) The patient's respiratory muscles ought to be fully relaxed during the EIP maneuver in order for the R and C computation to be valid.
2) The EIP maneuver is performed in a specific ventilation mode (Volume Assisted Control, VAC) and the resulting R and C values might not be representative of the corresponding values that determine the dynamics of the lung mechanics under other ventilation modes, such as PSV. Therefore, the accuracy of Pₘᵤₛ(t) computed via equation (1) during PSV operation can get compromised.
3) The EIP maneuver interrupts the regular ventilation pattern needed by the patient.

Finally, the above mentioned two-step techniques that apply LS fitting under specific conditions or to portions of the breath, where Pₘᵤₛ(t) is theoretically negligible, present limitations. In particular:
1) Repeated periods of paralysis, after the patient has recovered, plus CMV are not clinically feasible.
2) Repeated periods of high PSV interfere with the normal operation of the ventilator and may not be beneficial to the patient.
3) The assumption of negligible Pₘᵤₛ(t) during pressure-supported breaths is debatable, especially during the inhalation phase.

The present application provides new and improved systems and methods that facilitate noninvasively estimating of R, C, and Pₘᵤₛ using an airway occlusion pressure maneuver (P_{0.1}) having a predetermined duration, thereby overcoming the above-referenced problems and others.

### BRIEF SUMMARY

Still further advantages of the subject innovation will be appreciated by those of ordinary skill in the art upon reading and understand the following detailed description.

According to one embodiment, a method for estimating respiratory muscle pressure and respiratory mechanics using a P_{0.1} maneuver comprises detecting patient inspiration onset for a patient connected to a ventilator, occluding the airway of the patient for a first predetermined time period, and estimating a first respiratory muscle pressure (Pₘᵤₛ) profile during the airway occlusion. The method further comprises estimating resistance (R) and compliance (C) values and a second Pₘᵤₛ profile generated during a second predetermined time period, estimating a third Pₘᵤₛ profile during a third predetermined time period that extends from the end of the second predetermined time period until the end of the breath, and estimating Pₘᵤₛ(t) over an entire breath by concatenating the first, second and third Pₘᵤₛ profiles. The estimated R and C values and the estimated Pₘᵤₛ profiles are output on a display.

According to another embodiment, a system that facilitates estimating respiratory muscle pressure and respiratory mechanics using a P_{0.1} maneuver comprises a ventilator to which a patient is connected, and one or more processors configured to detect patient inspiration onset for a patient connected to a ventilator and occlude the airway of the patient for a first predetermined time period. The one or more processors are further configured to estimate a first respiratory muscle pressure (Pₘᵤₛ) profile during the airway occlusion, estimate resistance (R) and compliance (C) values and a second Pₘᵤₛ profile generated during a second predetermined time period, and estimate a third Pₘᵤₛ profile during a third predetermined time period that extends from the end of the second predetermined time period until the end of the breath. Additionally, the one or more processors are configured to estimate Pₘᵤₛ(t) over an entire breath by concatenating the first, second and third Pₘᵤₛ profiles, and output the estimated R and C values and the estimated Pₘᵤₛ profiles on a display.

According to another embodiment, a processor is configured to execute computer-executable instructions for estimating respiratory muscle pressure and respiratory mechanics using a P_{0.1} maneuver. The instructions comprise detecting processor patient inspiration onset for a patient connected to a ventilator, occluding the airway of the patient for a first predetermined time period, and estimating a first respiratory muscle pressure (Pₘᵤₛ) profile during the airway occlusion. The instructions further comprise estimating resistance (R) and compliance (C) values and a second Pₘᵤₛ profile generated during a second predetermined time period, and estimating a third Pₘᵤₛ profile during a third predetermined time period that extends from the end of the second predetermined time period until the end of the breath. Additionally, the instructions comprise estimating Pₘᵤₛ(t) over an entire breath by concatenating the first, second and third Pₘᵤₛ profiles, and outputting the estimated R and C values and the estimated Pₘᵤₛ profiles on a display.

### BRIEF DESCRIPTION OF THE DRAWINGS

The drawings are only for purposes of illustrating various aspects and are not to be construed as limiting.

FIG. 1 is a flowchart illustrating a method for estimating respiratory muscle pressure and respiratory mechanics using a P_{0.1} maneuver in accordance with one or more aspects described herein.

FIG. 2 illustrates a graph summarizing steps of the method of FIG. 1.

FIG. 3 illustrates exemplary results from method of FIG. 1 on one illustrative breath, where the estimated Pₘᵤₛ profile is compared against the gold-standard Pₘᵤₛ waveform measured inside the vessel.

FIG. 4 is a graph showing error that may be introduced during an occlusion period when a polynomial model of Pₘᵤₛ is fit to the airway pressure measurements during the occlusion period.

FIG. 5 illustrates a system that facilitates estimating respiratory muscle pressure and respiratory mechanics using a P_{0.1} maneuver in accordance with one or more aspects described herein.

FIG. 6 shows a system that facilitates estimating work of breathing (WOB) in a patient hooked up to a ventilator with automated software for the P_{0.1} maneuver.

FIG. 7 illustrates a system that facilitates estimating work of breathing (WOB) and power of breathing (POB) in a patient hooked up to a ventilator with automated software for the P_{0.1} maneuver wherein the ventilator is operating in Proportional Assist Ventilation (PAV) mode.

### DETAILED DESCRIPTION

The need for estimation of the respiratory system parameters (resistance R and compliance C) and patient inspiratory efforts (respiratory muscle pressure Pₘᵤₛ(t)) is well-known in the medical community. In order to overcome the above-described problems in the art, the herein-described systems and methods relate to an alternative approach for the noninvasive estimation of R, C, and Pₘᵤₛ that makes use of an airway occlusion pressure maneuver (P_{0.1}) having a predetermined duration (e.g., less than 150 ms or the like) to circumvent the inherent difficulty of the simultaneous estimation approach. The described method involves, inter alia, the following steps: 1) In the first step, the patient's airway is occluded at end of exhalation as soon as zero flow condition is detected; occlusion is maintained for a first predetermined time period, (e.g., 100 ms) and the airway pressure waveform during these 100 ms is used to estimate the coefficients of a polynomial model of Pₘᵤₛ(t); 2) once the occlusion has been released, the estimated Pₘᵤₛ(t) profile is extended (in time) for a second predetermined time period (e.g., for an additional 100 ms) and airway pressure and flow waveforms are used together with the extended Pₘᵤₛ profile to estimate R and C using the equation of motion via a standard Least-Square method; 3) the estimated R and C are used in conjunction with airway pressure and flow waveforms to reconstruct a Pₘᵤₛ profile for a third predetermined time period (e.g., throughout the remaining portion of the breath) based on the standard equation of motion. The P_{0.1} maneuver can be intermittently repeated at a variable or fixed rate (e.g., every X number of breaths) while the values of R and C estimated during the previous maneuver can still be used to compute an estimate of Pₘᵤₛ between each consecutive P_{0.1} maneuver. This also allows computation of WOB (or power of breathing (POB)) from the estimated Pₘᵤₛ profile on a breath-by-breath basis. In one embodiment, the claimed systems and methods are employed in hospital and home ventilators for real-time patient monitoring, ventilation optimization and closed-loop control.

The herein-described systems and methods overcome the aforementioned limitations of conventional approaches by; not requiring an esophageal balloon; explicitly accounting for the presence of Pₘᵤₛ; and not requiring a change ventilation mode during the maneuver so that the resulting R and C estimates are still related to the current ventilation operating conditions. Moreover, unlike the EIP, the P_{0.1} maneuver does not modify the patient's natural breathing pattern. Unlike other conventional approaches, the P_{0.1} is still reliable even when the ventilator cycles off before Pₘᵤₛ has returned to zero baseline value.

The described systems and methods facilitate performing noninvasive estimation of R, C and Pₘᵤₛ in patients receiving mechanical ventilation and able to breathe spontaneously. The R, C and Pₘᵤₛ estimates can be used for real-time patient monitoring, ventilation optimization and closed-loop control. The described systems and methods can be implemented as part of software or firmware running on a ventilator, anesthesia machines, or patient monitoring products (including remote patient monitors, e.g. eICU). The described systems and methods improve ventilator function by improving the accuracy of estimated R, C and Pₘᵤₛ values.

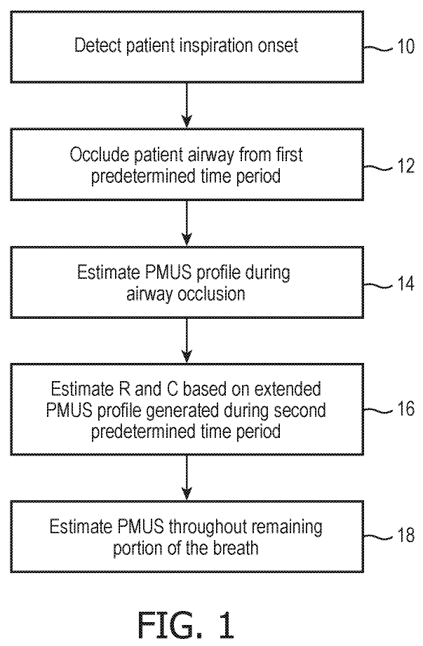

FIG. 1 is a flowchart illustrating a method for estimating respiratory muscle pressure and respiratory mechanics using a P_{0.1} maneuver in accordance with one or more aspects described herein. The method facilitates performing noninvasive estimation of R, C and Pₘᵤₛ(t) from airway pressure and flow measurements on a breath-by-breath basis. At 10, patient inspiration onset is detected, such as by sensing a characteristic pressure profile and flow profile from ventilator circuit pressure and flow sensors disposed in the patient circuit. At, 12, the patient's airway is occluded for a first predetermined time period by use of an occluder device such as a valve or flap disposed in the ventilator airflow path to the patient and under control of software for automatic operation. The first predetermined time period may be any suitable time period (e.g., less than approximately 150 ms, etc.). In the remainder of the document, a predetermined time period of 100 ms will be discussed but is not to be construed in a limiting sense. At 14, the initial inspiratory Pₘᵤₛ profile during airway occlusion is estimated. At 16, after the occlusion period, R and C are estimated based on an extended Pₘᵤₛ profile generated during a second predetermined time period. The second predetermined time period may be any suitable time period (e.g., less than approximately 150 ms, etc.) and need not be equal to the first predetermined time period in duration. At 18, Pₘᵤₛ is estimated using data collected during a third predetermined time period (e.g., throughout the remaining portion of the breath) following the second predetermined time period.

When performing estimation of the initial inspiratory Pₘᵤₛ profile during airway occlusion at 14, the patient's airway is occluded (at 12) at end of exhalation, as soon as the patient's inspiratory effort is detected (at 10). The occlusion is then maintained for, e.g., 100 ms, during which the patient is essentially trying to inhale against a closed airway. In one embodiment, the P_{0.1} maneuver is software-automated. During the occlusion, since there is no airflow between the point at which the airway pressure is measured and the patient's lungs, the negative deflection in pressure is measured at the airways (P_{aw}) and is essentially a reflection of the Pₘᵤₛ developed by the patient's respiratory muscles (gas decompression can be neglected) such that:

P_{aw}(t)=Pₘᵤₛ(t) for 0≤t≤100 ms

The small duration of the occlusion (e.g., less than 150 ms) ensures that the patient's natural respiratory Pₘᵤₛ output is not affected by the application of the occlusion. Hence, it is possible to fit a polynomial model of Pₘᵤₛ to the airway pressure measurements during the 100 ms occlusion and estimate the initial inspiratory Pₘᵤₛ profile via standard Least-Square (LS) technique. For instance, a 2^{nd} order polynomial Pₘᵤₛ model could be assumed and its unknown coefficients could then be estimated as shown below:

Pₘᵤₛ(t)=+a₂t+a₃·t² for 0≤t≤100 ms

$$Y = [ \begin{matrix}{P}_{aw} ( {t}_{1} ) \\ {P}_{aw} ( {t}_{2} ) \\ . \\ . \\ . \\ . \\ {P}_{aw} ( {t}_{k} )\end{matrix} ] = [ \begin{matrix}1 & {t}_{1} & {t}_{1}^{2} \\ 1 & {t}_{2} & {t}_{2}^{2} \\ & . & \\ & . & \\ & . & \\ & . & \\ 1 & {t}_{k} & {t}_{k}^{2}\end{matrix} ] [ \begin{matrix}{a}_{1} \\ {a}_{2} \\ {a}_{3}\end{matrix} ] = X \cdot θ$$

$$\hat{θ} = {[ {X}^{T} X ]}^{- 1} {X}^{T} Y$$

where θ is the vector of unknown parameters [a₁ a₂ a₃] (i.e., the coefficients of the polynomial Pₘᵤₛ model), Y is the vector containing the airway pressure measurements, k is the total number of samples collected during the 100 ms occlusion and t₁, t₂ tₖ are the time at which the airway pressure signal is sampled (i.e., t₁=0, t₂=T, t₃=2T, . . . tₖ=(k−1)T with T being the sample period).

When estimating R and C after the occlusion based on a 100 ms extended Pₘᵤₛ profile at 16, after the 100 ms occlusion period, the airways are released and air flows to the lungs under the pressure gradient established by both the patient's own Pₘᵤₛ drive and the contribution of the ventilator. In such conditions, it may be difficult to estimate R, C and Pₘᵤₛ simultaneously from flow and pressure measurements based on the simple equation of motion because the underlying LS problem is underdetermined. However, it is reasonable to assume that for a very short period of time (e.g., 100 ms), the profile of Pₘᵤₛ remains unchanged compared to the profile estimated during the previous 100 ms occlusion period. Hence, it is possible to extend the Pₘᵤₛ profile based on the previously estimated polynomial coefficients and attain an estimate of Pₘᵤₛ during this additional 100 ms post-occlusion period such that:

\hat{P}ₘᵤₛ(t)=a₁+a₂·t+a₃·t² for 100 ms≤t≤200 ms

The extended Pₘᵤₛ(t) profile can be used together with the airway pressure and flow waveforms to estimate R and C using the equation of motion via a LS method such that:

$$\begin{matrix}{P}_{aw} ( t ) - {\hat{P}}_{mus} ( t ) = \hat{R} \cdot \dot{V} ( t ) + \hat{E} \cdot V ( t ) + {P}_{0} for 100 ms \leq t \leq 200 ms \\ \overline{Y} = [ \begin{matrix}{P}_{aw} ( {t}_{k + 1} ) - {\hat{P}}_{mus} ( {t}_{k + 1} ) \\ {P}_{aw} ( {t}_{k + 2} ) - {\hat{P}}_{mus} ( {t}_{k + 2} ) \\ . \\ . \\ . \\ . \\ {P}_{aw} ( {t}_{k + K} ) - {\hat{P}}_{mus} ( {t}_{k + K} )\end{matrix} ] = [ \begin{matrix}\dot{V} ( {t}_{k + 1} ) & V ( {t}_{k + 1} ) & 1 \\ \dot{V} ( {t}_{k + 2} ) & V ( {t}_{k + 2} ) & 1 \\ & . & \\ & . & \\ & . & \\ & . & \\ \dot{V} ( {t}_{k + K} ) & V ( {t}_{k + K} ) & 1\end{matrix} ] [ \begin{matrix}R \\ E \\ {P}_{0}\end{matrix} ] = \overline{X} \cdot \overline{θ} \\ \hat{\overline{θ}} = {[ {\overline{X}}^{T} \overline{X} ]}^{- 1} {\overline{X}}^{T} \overline{Y}\end{matrix}$$

where P_{aw}(t) is the pressure measured at the airway opening, \dot{V}(t) is the flow of air into and out of the patient's respiratory system (measured again at the airway opening), V(t) is the net volume of air delivered to the patient (measured by integrating the flow signal over time), E is the elastance (inverse of the compliance C), P₀ is a constant term to account for the pressure at the end of expiration (needed to balance the equation but not interesting per se), θ̅ is the vector of unknown parameters [R E P₀], K is the number of samples collected during the 100 ms post-occlusion period, and tₖ₊₁, tₖ₊₂ . . . t_{K+K} are the times (within the 100 ms post-occlusion period) at which airway pressure and flow signals are sampled.

When estimating Pₘᵤₛ throughout the remaining portion of the breath at 18, the values of R and C computed during the previous step are used in conjunction with airway pressure and flow waveforms to compute an estimate of Pₘᵤₛ throughout the remaining portion of the breath based on the standard equation of motion such that:

\hat{P}ₘᵤₛ(t)=P_{aw}(t)−\hat{R}·\dot{V}(t)−Ê·V(t)−P₀ for 200 ms≤t≤t_{end} (2)

where t_{end} is the last available time sample (time at the end of the breath).

While the systems and methods discussed herein have been described with respect to certain embodiments, it is to be understood that said systems and methods are not limited to the disclosed embodiments and examples. To the contrary, described systems and methods are intended to cover various modifications and equivalent arrangements included within the spirit and scope of the described algorithm. For instance: The degree of the polynomial Pₘᵤₛ model used in step 14 of FIG. 1 (occlusion period) can be different than 2. For instance, a first order polynomial model (i.e., a line) could also be used.

In another embodiment, the duration of step 16 (post-occlusion period) is not limited to being 100 ms. A short duration is useful in order for the assumption of unaltered Pₘᵤₛ profile from step 14 to step 16 to be as valid as possible. In fact, the initiation of pressurization, provided by the ventilator after release of the airways occlusion, can induce changes in the patient's own Pₘᵤₛ drive via mechanical reflexes (e.g. Hering-Breuer reflex). However, the activation of such reflexes and the manifestation of their effects on Pₘᵤₛ may occur on a time scale that is larger than 100 ms. On the other hand, a too short duration of step 16 may induce noise in the measurements that could compromise the LS procedure and lead to biased R and C estimates.

The final estimated Pₘᵤₛ profile does not necessarily need to be constructed by concatenating the three Pₘᵤₛ profiles obtained during steps 14, 16, and 18, respectively. According to one embodiment, the values of R and E, which is essentially the inverse of C, from step 16 are used to compute the estimated Pₘᵤₛ profile over the entire breath according to:

\hat{P}ₘᵤₛ(t)=P_{aw}(t)−\hat{R}·\dot{V}(t)−Ê·V(t)−P₀ for 0≤t≤t_{end} (3)

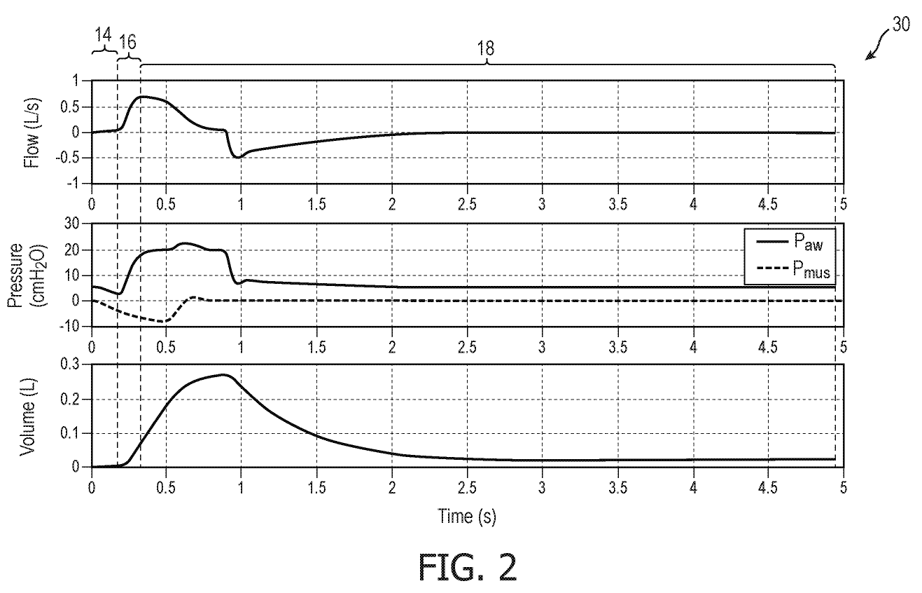

FIG. 2 illustrates a graph 30 summarizing steps 14, 16, and 18 of the method of FIG. 1. Exemplary results of the herein-described estimation algorithm have been generated using an in-vitro hydraulic model of the lungs. The in-vitro model was comprised of a rigid vessel within which an elastic balloon was placed. The balloon was characterized by a certain elastance value and its behavior was approximated as linear within a certain range of pressure values. The system was connected to a mechanical ventilator (e.g., Esprit, Philips-Respironics) via a linear resistor. The pressure within the vessel and external to the balloon was artificially controlled via an automated vacuum and compressed air system. Hence, a specific nominal Pₘᵤₛ profile can be generated externally to the balloon. The ventilator was then operated in Pressure Control Mode (note that any other suitable modes can be selected) and a P_{0.1} maneuver was performed via the automatic software embedded in the ventilator. Pressure and flow measurements were collected via dedicated sensors placed at the Y connection between the ventilator and the in-vitro lung model.

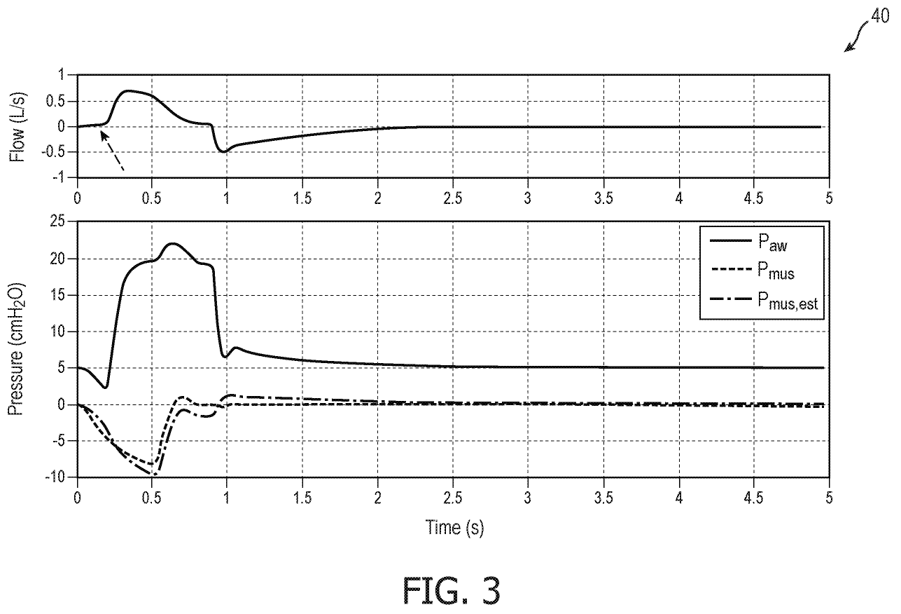

FIG. 3 illustrates exemplary results 40 from method of FIG. 1 on one illustrative breath, where the estimated Pₘᵤₛ profile is compared against the gold-standard Pₘᵤₛ waveform measured inside the vessel. The level of agreement between the two waveforms is acceptable in this example (RMSE=0.7297) even though a certain degree of error can be observed. Furthermore, the R and E estimates provided by the algorithm (\hat{R}=24.85, Ê=56.84) are very close to the corresponding gold standard values (R_{gs}=22.35, E_{gs}=54.11) computed via LS using the gold standard Pₘᵤₛ waveform.

The small error between the Pₘᵤₛ, R and E estimates and the corresponding gold standard values can be in part ascribed to the existence of a non-zero flow during the occlusion (see dashed arrow in FIG. 3). This non-zero flow is essentially due to decompression of the air contained within the system when both the inhalation and the exhalation valves are closed. In fact, due to non-zero flow during the occlusion, Eq. (1) is no longer valid:

P_{aw}(t)≠Pₘᵤₛ(t) for 0≤t≤100 ms

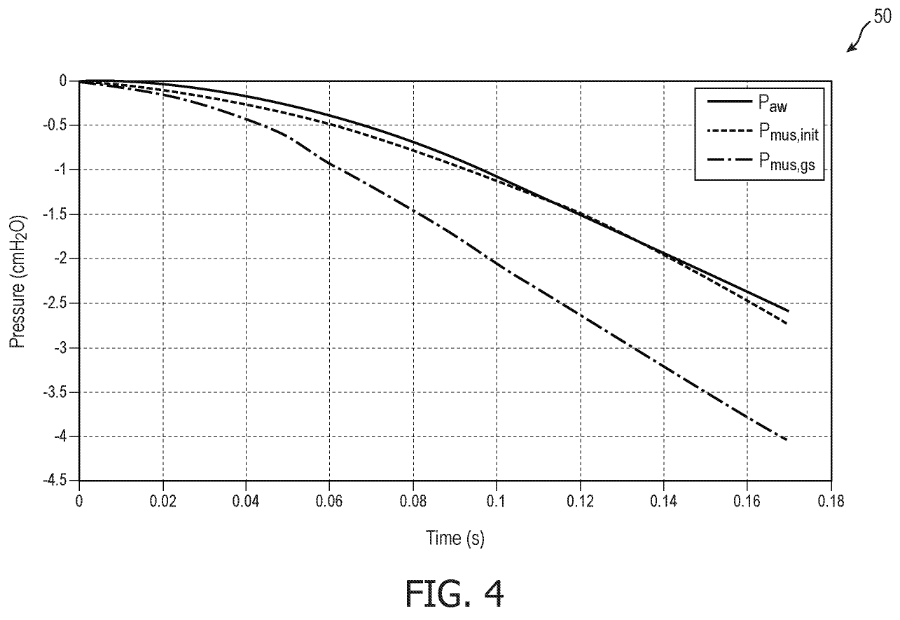

Hence, when a polynomial model of Pₘᵤₛ is fit to the airway pressure measurements during the occlusion period, error may be introduced as shown in the graph 50 of FIG. 4. This error can be mitigated by reducing the length of the tubing circuit thus reducing the magnitude of the non-zero flow during the occlusion.

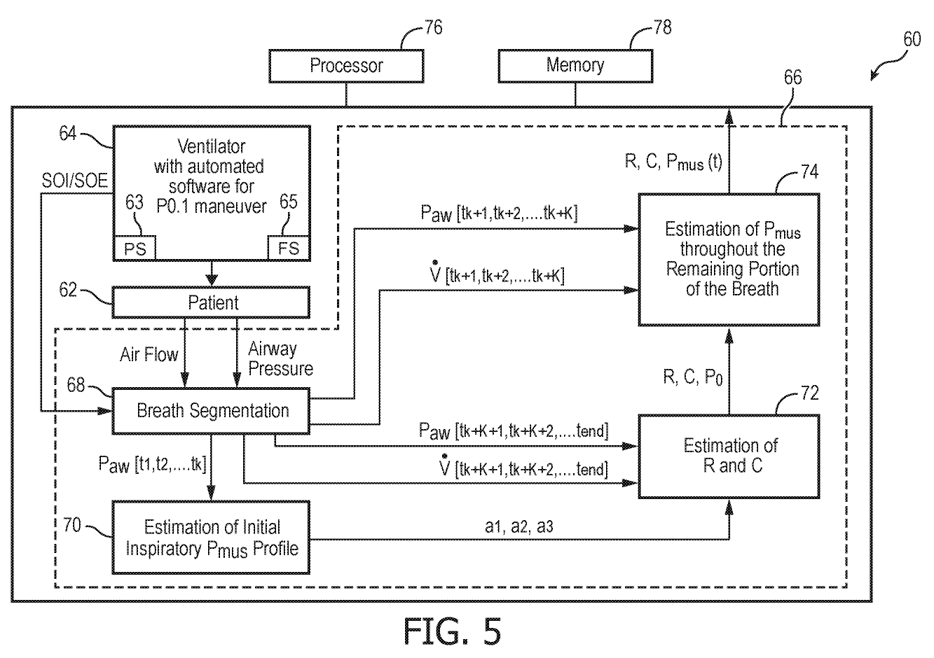

FIG. 5 illustrates a system 60 that facilitates estimating respiratory muscle pressure and respiratory mechanics using a P_{0.1} maneuver in accordance with one or more aspects described herein. According to the embodiment of FIG. 5, a patient 62 is connected to a ventilator 64 having one or more pressure sensors 63 and one or more flow sensors 65 in the patient circuit that respectively sense a characteristic pressure profile and a flow profile in the patient circuit. The ventilator is equipped with software and/or hardware configured to perform automatically a P_{0.1} maneuver. The airway pressure and flow signals are measured in real-time; for example, volume can be computed by numerical integration of the flow signal. The system further comprises an estimation module 66 that includes a breath segmentation algorithm or module 68 that is used to isolate the current breath, starting from the moment at which the airways are occluded (e.g., at the beginning of patient's inspiratory activity) to the moment at which the exhalation is completed. To do so, specific flags from the ventilator (e.g. start of inspiration (SOI), start of expiration (SOE), etc.) are used. These flags contain the timestamps of the opening and closing of the inhalation and exhalation valves. The breath segmentation algorithm also divides the airflow and pressure data from the current breath into 3 different subsets related to the 3 regions identified in FIG. 2, e.g.: 1) 100 ms occlusion region; 2) 100 ms post-occlusion region; 3) remaining portion of the breath. Flow and pressure data from the 3 segmented breath regions are provided as input to 3 estimation routines or modules, including a Pₘᵤₛ profile estimation routine or module 70, an R and C estimation routine or module 72, and a Pₘᵤₛ remainder-of-breath (ROB) routine or module 74 for estimating Pₘᵤₛ throughout the remaining portion of the breath. Each routine executes one of the 3 aforementioned estimation steps 14, 16, 18 of FIG. 1 sequentially. Once the three steps have been executed, an estimate of Pₘᵤₛ(t) over the entire breath can be computed by concatenating the 3 Pₘᵤₛ(t) profiles computed during each step 14, 16, and 18. Finally, the R, C and Pₘᵤₛ(t) estimated by the algorithm are provided as output. These can be displayed directly on the ventilator screen, or on a separate patient monitor.

The estimation algorithm 66 illustrated in the embodiment of FIG. 5 can run on a ventilator processor or on a separate patient monitor. Additionally, the estimation algorithm 66 can run continually on a breath-by-breath basis, and a P_{0.1} maneuver can be performed at every breath. This allows breath-by-breath updating of the estimated R and C and tracking of potential changes in patients' respiratory mechanics from one breath to the next one. Alternatively, the P_{0.1} maneuver can be performed intermittently (e.g., at every X number of breaths, where X is an integer), while the values from the latest R and C estimation procedure are assumed valid over the next subsequent breaths and used to compute an estimate of Pₘᵤₛ(t) based on the equation of motion (as in Eq. 2) until a new P_{0.1} maneuver is performed.

The system further comprises a processor 76 that executes, and a memory 78 that stores, computer executable instructions for carrying out the various functions and/or methods described herein. The memory 78 may be a computer-readable medium on which a control program is stored, such as a disk, hard drive, or the like. Common forms of computer-readable media include, for example, floppy disks, flexible disks, hard disks, magnetic tape, or any other magnetic storage medium, CD-ROM, DVD, or any other optical medium, RAM, ROM, PROM, EPROM, FLASH-EPROM, variants thereof, other memory chip or cartridge, or any other tangible medium from which the processor 76 can read and execute. In this context, the described systems may be implemented on or as one or more general purpose computers, special purpose computer(s), a programmed microprocessor or microcontroller and peripheral integrated circuit elements, an ASIC or other integrated circuit, a digital signal processor, a hardwired electronic or logic circuit such as a discrete element circuit, a programmable logic device such as a PLD, PLA, FPGA, Graphics processing unit (GPU), or PAL, or the like.

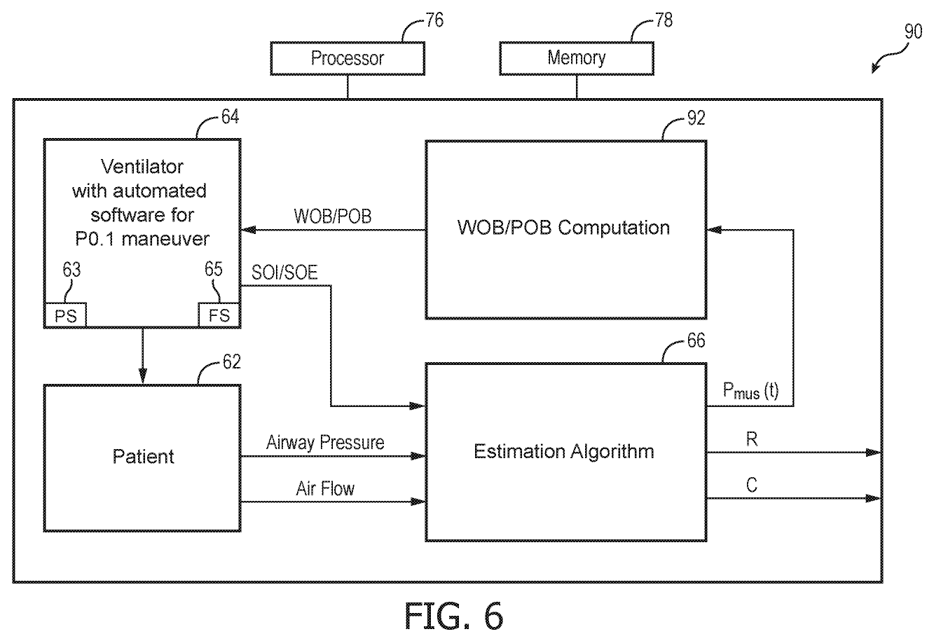

FIG. 6 shows a system 90 that facilitates estimating work of breathing (WOB) in a patient 62 hooked up to a ventilator 64 having one or more pressure sensors 63 and one or more flow sensor 65 and equipped with automated software for the P_{0.1} maneuver. The Pₘᵤₛ output from the estimation algorithm 66 is used at WOB estimating step 92 to compute an estimate the Work of Breathing (WOB) by integrating the product between Pₘᵤₛ(t) and \dot{v}(t) over the inhalation phase of the current breath. From the computed WOB, Power of Breathing (POB) can also be attained by summing WOB over one minute. The estimated WOB/POB can ultimately be displayed on the ventilator screen or used internally by the ventilator as input to a closed-loop controller. The system further comprises a processor 76 that executes, and a memory 78 that stores, computer executable instructions for executing the various modules, algorithms, routines, etc. of FIG. 6.

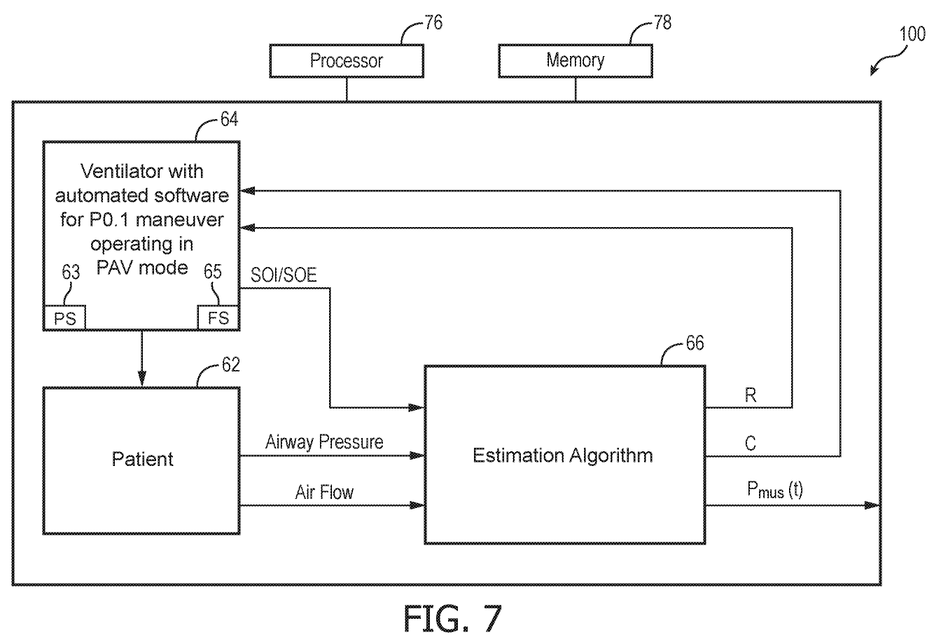

FIG. 7 illustrates a system 100 that facilitates estimating work of breathing (WOB) and power of breathing (POB) in a patient 62 hooked up to a ventilator 64 with automated software for the P_{0.1} maneuver wherein the ventilator is operating in Proportional Assist Ventilation (PAV) mode. The ventilator further comprises one or more pressure sensors 63 and one or more flow sensors 65 in the patient circuit that respectively sense a characteristic pressure profile and a flow profile in the patient circuit. The R and C values estimated by the algorithm 66 can be used to compute a desired airway pressure signal proportional to Pₘᵤₛ and drive the mechanical ventilator in PAV mode. The system further comprises a processor 76 that executes, and a memory 78 that stores, computer executable instructions for executing the various modules, algorithms, routines, etc. of FIG. 6.

The innovation has been described with reference to several embodiments. Modifications and alterations may occur to others upon reading and understanding the preceding detailed description. It is intended that the innovation be construed as including all such modifications and alterations insofar as they come within the scope of the appended claims or the equivalents thereof.

1. A method for estimating respiratory muscle pressure and respiratory mechanics using a P_{0.1} maneuver for a patient connected to a ventilator, comprising:
detecting patient inspiration onset;
automatically occluding the airway between the ventilator and the patient for a first predetermined time period responsive to the detecting step,
estimating a first respiratory muscle pressure (Pₘᵤₛ) profile during the airway occlusion;
estimating resistance (R) and compliance (C) values and a second Pₘᵤₛ profile generated during a second predetermined time period;
estimating a third Pₘᵤₛ profile during a third predetermined time period that extends from the end of the second predetermined time period until the end of inspiration;
estimating Pₘᵤₛ(t) over an entire breath by concatenating the first, second and third Pₘᵤₛ profiles; and
outputting the estimated R and C values and the estimated Pₘᵤₛ profiles on a display.

2. The method according to claim 1, wherein estimating the first Pₘᵤₛ profile during the airway occlusion comprises fitting a first polynomial model of Pₘᵤₛ to the airway pressure measurements during the airway occlusion and estimating the first Pₘᵤₛ profile via a Least-Square (LS) technique.

3. The method according to claim 2, wherein estimating the second Pₘᵤₛ profile during the airway occlusion comprises extending the first polynomial model of Pₘᵤₛ in time.

4. The method according to claim 1, wherein the first and second time periods are less than approximately 150 ms in duration.

5. The method according to claim 1, wherein the first and second time periods are approximately 100 ms in duration.

6. The method according to claim 1, further comprising estimating a work of breathing (WOB) by integrating a product between Pₘᵤₛ(t) and \dot{V}(t) over an inhalation phase of a current breath.

7. The method according to claim 1, wherein the ventilator is operating in proportional assist ventilation (PAV) mode, and further comprising computing a desired airway pressure signal proportional to Pₘᵤₛ for driving the ventilator in PAV mode.

8. A processor or computer-readable medium having stored thereon computer-readable instructions for performing the method of claim 1.

9. A system that facilitates estimating a patient's respiratory muscle pressure and respiratory mechanics using a P_{0.1} maneuver, comprising:
a ventilator having a pressure sensor and a flow sensor; and
one or more processors in communication with the ventilator and configured to:
detect patient inspiration onset for a patient connected to the ventilator;
automatically occlude the airway of the patient for a first predetermined time period responsive to the detect,
estimate a first respiratory muscle pressure (Pₘᵤₛ) profile during the airway occlusion;
estimate resistance (R) and compliance (C) values and a second Pₘᵤₛ profile generated during a second predetermined time period;
estimate a third Pₘᵤₛ profile during a third predetermined time period that extends from the end of the second predetermined time period until the end of inspiration;
estimate Pₘᵤₛ(t) over an entire breath by concatenating the first, second and third Pₘᵤₛ profiles; and
output the estimated R and C values and the estimated Pₘᵤₛ profiles on a display.

10. The system according to claim 9, wherein the one or more processors are further configured to estimate the first Pₘᵤₛ profile during the airway occlusion by fitting a polynomial model of Pₘᵤₛ to the airway pressure measurements during the airway occlusion and estimate the initial inspiratory Pₘᵤₛ profile via a Least-Square (LS) technique.

11. The system according to claim 9, wherein the first and second time periods are less than approximately 150 ms in duration.

12. The system according to claim 9, wherein the first and second time periods are approximately 100 ms in duration.

13. The system according to claim 9, wherein the one or more processors are further configured to estimate a work of breathing (WOB) by integrating a product between Pₘᵤₛ(t) and \dot{V}(t) over an inhalation phase of a current breath.

14. The system according to claim 9, wherein the ventilator is operating in proportional assist ventilation (PAV) mode.

15. The system according to claim 14, he one or more processors are further configured to compute a desired airway pressure signal proportional to Pₘᵤₛ for driving the ventilator in PAV mode.

16.-20. (canceled)
